(19) United States
(12) Patent Application Publication
(10) Pub. No.: US 2005/0069584 A1
(43) Pub. Date: Mar. 31, 2005

(54) Diphenhydramine tannate solid dose compositions and methods of use

(76) Inventors: Jeffrey S Kiel, Gainesville,, GA (US); H Greg Thomas, Villa Rica, GA (US); Narasimhan Mani, Port Jefferson, NY (US)

Correspondence Address: KING & SCHICKLI, PLLC, 247 NORTH BROADWAY, LEXINGTON, KY, 40507, US

(21) Appl. No.: 10/505,347

(22) PCT Filed: Feb. 26, 2003
(86) PCT No.: PCT/US03/05664

(30) Foreign Application Priority Data
Apr. 9, 2002 (US) 10119285
Oct. 10, 2002 (US) 10269027

(51) Int. Cl. A61K 31/7024; A61K 31/205; A61K 9/20

(52) U.S. Cl. 424/464; 514/23; 514/554

(57) ABSTRACT

Pharmaceutical compositions consisting of diphenhydramine tannate in solid dosage form which are effective when administered for the symptomatic relief of sneezing, itchy, watery eyes, itchy nose or throat and runny nose due to hay fever (allergic rhinitis) or other respiratory allergies are disclosed.

This is a continuation-in-part of U.S. patent application Ser. No. 10/119,285 filed Apr. 9, 2002 which claims the benefit of Provisional Patent Application Ser. No. 60/282,969 filed Apr. 10, 2001 and U.S. patent application Ser. No. 10/269,027 filed Oct. 10, 2002 which claims the benefit of Provisional Patent Application Ser. No. 60/328,990 filed Oct. 12, 2001.

### FIELD OF INVENTION

The invention relates to novel antihistaminic tannate compositions. The compositions contain as an essential ingredient diphenhydramine tannate.

### BACKGROUND OF THE INVENTION

Tannins are water-soluble phenolic metabolites of plants with a molecular weight of 5-5000 Da. Physicochemically, tannins are complex polymers, which can be classified as two major types: the condensed tannins and hydrolyzable tannins. Hydrolyzable tannins or tannic acids are referenced in the various pharmacopeias and are composed of gallic acid or its condensation product ellagic acid esterified to the hydroxyl groups of glucose. Each hydrolyzable tannin molecule is usually composed of a core D-glucose and 6 to 9 galloyl groups.

In nature, there is an abundance of mono and di-galloyl esters of glucose with a molecular weight of about 900. These are not considered to be tannins. At least 3 hydroxyl groups of the glucose must be esterified to exhibit a sufficiently strong binding capacity to be classified as tannin.

Tannic acid, also known as tannin, is commercially available with a water content of about 5% to about 10% by weight and a molecular weight of about 1700. It is typically produced from Turkish or Chinese nutgall and has a complex, non-uniform chemistry.

Diphenhydramine is known chemically as 2-(benzhydroxyl)-N,N-dimethylethylamine. The methods of preparation of the drug are described in U.S. Pat. Nos. 2,421,714 and 2,397,799. Diphenhydramine Hydrochloride salt has a melting point of 166-170 degrees C. and is soluble in water and sparingly soluble in alcohol. The pH of a 1% aqueous solution is about 5.5. Diphenhydramine belongs to the class of ethanolamine H1 receptor blockers, and possesses in addition to antihistaminic activity, a significant anticholinergic effect, which makes it highly effective for the symptomatic relief of sneezing, itchy, watery eyes, itchy nose or throat and runny nose due to hay fever (allergic rhinitis) and other respiratory allergies. It has lower incidences of gastrointestinal side effects than compositions containing other antihistamine compounds by themselves or in combination with diphenhdyramine. Diphenhydramine also possesses a pronounced tendency to induce sedation.

Antihistamine compounds in the form of their free bases as well as their salts, e.g. hydrochloride, maleate, tannate, etc. are well known. Frequently it is desirable to utilize the antihistamine in the form of its tannate salt, because such salt is generally quite stable and may be administered in such form without any side effects. In addition, the tannate salt of the active is a significantly larger molecule, which affords absorption of the active over prolonged intervals of time, reducing the sedative action, frequency of administration and thereby improves patient compliance in comparison to other salt forms of antihistamines.

Antihistamines in the form of their tannate salts can be prepared by following a number of different procedures. In a first approach, the free base, e.g. diphenhydramine, etc. is reacted with tannic acid in the presence of a volatile solvent, isopropanol. Typically, in the conventional isopropanol route, the antihistaminic free base and the tannic acid will be present in the isopropanol at a concentration based on the weight of the reaction mixture. The reaction mixture is stirred for about one hour while maintaining the mixture at 60-70 degrees C. The reaction mixture is cooled to room temperature and then filtered, washed with isopropanol and then vacuum dried. However, antihistamine tannate salts are heat sensitive and therefore undergo decomposition quite readily upon prolonged exposure to temperatures as low as 50 degrees C. In addition, the yield obtained is usually only about 70% and impurities including decomposition products and a significant amount of the volatile solvent used during preparation (up to about 5-10%) cannot be effectively removed. Decomposition products of diphenhydramine resulting from exposure to higher temperatures associated with this first production approach include benzhydrol, benzophenone, diphenylchloromethane, dimethylaminoethanol, diphenylmethane, diphenyl alky ether and mixtures thereof.

Further, due to the large size of the tannate molecule, the percentage of active free-base within the tannate salt is significantly lower than that in other salt forms such as the hydrochloride or maleate. The low active percentages and the variable purity of the tannate compounds prepared by these synthetic methods leads to a stoichiometry of the active free base to tannic acid in the tannate salt to be different from batch to batch. This causes significant problems during manufacture of products containing tannate salts as active ingredients and increases the likelihood that commercially available pharmaceutical products contain variable and in some instances, sub-therapeutic levels of the active drug substances creating dosage problems.

A second approach to prepare the antihistamine tannates, is to contact the free base form of the drug with tannic acid in the presence of water for a suitable period of time and at a maximum temperature. The antihistamine tannate salt is usually isolated and purified by freeze-drying and then subsequently introduced into pharmaceutically effective dosage forms. This approach results in a dosage form suffering from a number of shortcomings. These include the use of expensive equipment and the time involved in freeze-drying. This approach also suffers from batch to batch variability and all the attendant disadvantages outlined above. Further, the development of a suitable and effective freeze drying process can be complicated.

A third and better approach to prepare the antihistamines in the form of their tannate salts is disclosed in our copending U.S. patent application Ser. No. 10/269,027 filed Oct. 10, 2002, entitled “Process For Preparing Tannate Tablet, Capsule Or Other Dosage Forms”, the full disclosure of which is incorporated herein by reference. In this approach, an aqueous solution or the powder form of the drug is reacted with a tannic acid mixture in liquid or powder form. The tannate salt prepared by this method can be isolated and purified by filtration, drying or centrifugation or can be directly incorporated into suitable pharmaceutically effective dosage forms without the need for further isolation or purification. In addition, the exposure of tannate salts to high temperatures that can produce undesirable decomposition products is also avoided.

The tannate salt of the antihistamine can also be prepared without the use of organic solvents, which would be desirable from an environmental standpoint. This also allows one to eliminate organic solvents as a possible contaminant in the final dosage product. In addition, a commercially available USP/NF grade salt or the free base of the antihistamine can be used with USP/NF grade tannic acid to prepare the tannate salt. This insures that the stoichiometry of the active ingredient may be properly matched to the tannic acid. As a result, the potency of the finished product is less variable and, therefore, more precise dosing is possible. Patient benefits include more effective treatment with minimal unwanted or adverse side effects.

### SUMMARY OF THE INVENTION

The present invention relates to a therapeutic composition for symptomatic treatment of respiratory allergies in a warm-blooded animal where that composition comprises a pharmaceutically effective amount of diphenhydramine tannate at a consistent purity in the substantial absence of an organic solvent in solid dosage form. That organic solvent may, for example, be a mineral oil or an alcohol including but not limited to such solvents as isopropyl alcohol, glycerine, propylene glycol and ethanol.

Alternatively, the invention may be described as a therapeutic composition for symptomatic treatment of respiratory allergies in a warm-blooded animal where the composition comprises a pharmaceutically effective amount of diphenhydramine tannate at a consistent purity in substantial absence of decomposition products of diphenhydramine tannate produced at temperatures above about 50 degrees C. in solid dosage form. Such decomposition products include but are not necessarily limited to benzhydrol, benzophenone, diphenylchloromethane, dimethylaminoethanol, diphenylmethane and diphenyl alkyl ether.

Still further the therapeutic composition may be defined as comprising a pharmaceutically effective amount of diphenhydramine tannate at a consistent purity prepared in a preferred way by:
(a) dissolving the salt or free base of the diphenhydramine in a pharmaceutically acceptable liquid to form a solution at a maximum temperature and pH value, that does not cause decomposition of the active pharmaceutical ingredient;
(b) separately mixing an anti-clumping agent with tannic acid to generate a blend;
(c) combining the solution from step (a), with the blend of step (b) to form a tannate salt of diphenhydramine;
(d) combining the tannate salt of the diphenhydramine of step (c) with a pharmaceutically acceptable excipient to form a granulate; and
(e) processing the granulate into a tablet, capsule or other solid dosage form.

In accordance with yet another aspect of the present invention, a method is provided for symptomatically treating respiratory allergies in a warm-blooded animal. That method comprises administering to the warm-blooded animal a pharmaceutically effective amount of diphenhydramine tannate at a consistent purity in substantial absence of an organic solvent in a solid dosage form.

Alternatively, the method may be described as comprising administering to the warm-blooded animal a pharmaceutically effective amount of diphenhydramine tannate at a consistent purity in substantial absence of decomposition products of diphenhydramine tannate produced at temperatures above about 50 degrees C. in a solid dosage form.

Still further the method may be described as comprising administering to the warm-blooded animal a pharmaceutically effective amount of diphenhydramine tannate at a consistent purity prepared by:
(a) dissolving the salt or free base of the diphenhydramine in a pharmaceutically acceptable liquid to form a solution at a maximum temperature and pH value, that does not cause decomposition of the active pharmaceutical ingredient;
(b) separately mixing an anti-clumping agent with tannic acid to generate a blend;
(c) combining the solution from step (a), with the blend of step (b) to form a tannate salt of diphenhydramine;
(d) combining the tannate salt of the diphenhydramine of step (c) with a pharmaceutically acceptable excipient to form a granulate; and
(e) processing the granulate into a tablet, capsule or other solid dosage form.

### DETAILED DESCRIPTION OF THE INVENTION

The present invention relates to a novel therapeutic composition in solid dosage form containing a tannate salt of the active ingredient diphenhydramine at a consistent purity. The composition is useful for the treatment of symptoms of sneezing, itchy, watery eyes, itchy nose or throat and runny nose due to hay fever (allergic rhinitis) or other respiratory allergies.

The compositions may be prepared for oral administration in the form of powders, capsules, and the preferred forms of tablets formulated so that ideally each tablet contains about 0.1 mg to about 300 mg, preferably about 25 mg of diphenhydramine tannate.

A preferred way of preparing the tannate salt is by reacting the aqueous solution or the powder form of the drug with a tannic acid mixture in liquid or powder form without the use of volatile solvents. The tannate salt prepared is then directly incorporated into suitable pharmaceutically effective dosage forms without further purification and isolation.

Stated another way, the first step of this process is to create a tannic acid powder blend by combining the active pharmaceutical ingredient (API) diphenhydramine with tannic acid in the presence of a pharmaceutically acceptable liquid. An anti-clumping agent also may be added to the mix. The presence of the anti-clumping agent prevents the aggregation of the tannate salt formed and promotes uniformity in the powder blend.

The source of the tannic acid is natural or synthetic. The formation of the tannate salt is by reaction of amine groups (in the 1°, 2°, 3°, 4°, or amphoteric functional states) or of the other basic functional groups with tannic acid. The amount and ratio of dispersing agent and tannic acid required for the completion of the reaction is determined by the molecular configuration and concentration of the diphenhydramine.

The tannate salt obtained from the above-conversion process is mixed with a diluent, binder(s), lubricants, sweetening, hardness increasing, coloring, flavoring and flow agents as necessary. The resulting granulate is processed into tablet, capsule or other solid-dosage forms as necessary.

By starting with a known amount of commonly available salt or the free base form of the diphenhydramine, which is subsequently converted and incorporated as a tannate salt into a solid dosage form, the invention provides an efficient and reproducible method to manufacture products containing diphenhydramine tannate salt as an active ingredient. Since the tannate salt of the diphenhydramine is generated and incorporated into the dosage form during the manufacturing process, the need to isolate the tannate salt is eliminated and the stoichiometry of the tannate salt is uniform from batch to batch. Thus, for the first time the diphenhydramine tannate is provided at a consistent purity. This is particularly true when using USP/NF grade starting materials.

The excipients commonly used in the formulations are as follows: Microcrystalline cellulose (Avicel), lactose, Mannitol and Di-Pac (compressible sugar) as diluents; magnesium aluminum silicate, xanthan gum, polyvinylpyrrolidone and cellulose compounds as anti-clumping agents; starch hydroxypropyl methylcellulose (HPMC E-10) and xanthan gum as binders; sweetening agents such as sucrose, saccharin sodium, Sucralose and Magnasweet; calcium phosphate as hardness enhancer; talc as a glidant and magnesium stearate as a lubricant. Active ingredients not present as tannate salts also can be included in the formulation.

The diphenhydramine salts of the active ingredients are preferably dissolved in purified water. This leads to the dissociation of the salt into its free-base and conjugate acid forms. During the synthesis process the pH is maintained between about 2 to about 11 while not exceeding a temperature of about 15 to about 40 degrees C. so as to minimize or substantially avoid the production of decomposition products.

The following EXAMPLES illustrate the conversion process and subsequent incorporation of the tannate salts into suitable solid dosage forms.

### EXAMPLE 1

### Preparation Of A Dosage Form With One API

| Ingredient | Amount (g) | |
|---|---|---|
| diphenhydramine HCl | 12.500 | |
| tannic acid | 32.813 | |
| purified water | 12.5 | mL |

The ingredients used in the conversion process to generate 25 g of diphenhydramine as the tannate salt are shown above. Diphenhydramine hydrochloride and tannic acid are placed in a suitable planetary mixer or blender and the powders are mixed for a period of ten minutes to obtain a uniform powder blend of the ingredients. Once the powders are mixed and a uniform blend obtained, the water is sprayed onto the mixing powders and mixing is continued for ten to fifteen minutes to generate the tannate salt of diphenhydramine. The synthetic process yields diphenhydramine tannate salt as a uniformly distributed powder mass. The weight ratio of diphenhydramine to tannic acid used is 1:3.

The powder mass of the tannate salt obtained from the conversion step is used as is for incorporation into capsules or subsequently can be dried and blended with more diluent, hardness increasing and coloring agents as necessary to form a tablet. A typical tablet prepared by well known conventional manufacturing techniques is shown below.

| Ingredient | mg/tablet |
|---|---|
| diphenhydramine tannate* | 25.000 |
| magnesium aluminum silicate, NF | 6.750 |
| Avicel PH 102 | 157.181 |
| Sodium Saccharin | 4.500 |
| 15 Methocel E-10M | 6.750 |
| corn starch | 4.500 |
| Di-Pac | 244.175 |
| calcium phosphate dibasic | 13.500 |
| xanthan gum | 7.875 |
| strawberry flavor | 4.500 |
| talc | 2.250 |
| FD&C Blue No. 2 | 0.450 |
| magnesium stearate | 2.250 |

*equivalent to 12.5 mg diphenhydramine HCl

### EXAMPLE 2

### Preparation Of A Capsule Dosage Form With Two APIs

The powder mass of the tannate salts of the two APIs obtained from the conversion step are mixed with a diluent, flow agents and lubricants. The powder mixture can subsequently be filled into size 1 capsules. A typical capsule formulation prepared by well known conventional encapsulation techniques is shown below.

| Ingredient | mg/capsule |
|---|---|
| diphenhydramine tannate* | 25.000 |
| phenylephrine tannate** | 12.500 |
| PVP | 20.000 |
| Mannitol | 528.000 |
| talc | 2.250 |
| magnesium stearate | 2.50 |

*equivalent to 12.5 mg of diphenhydramine HCl
**equivalent to 2.5 mg phenylephrine HCl
The ratio of diphenhydramine to tannic acid in the tannate salt is 1:1.3 and phenylephrine to tannic acid is 1:2, by weight.

### EXAMPLE 3

### Preparation Of A Capsule Dosage Form With Three Or More APIs

The conversion process to generate the tannate salts can be performed by using a powder blend to which the solutions of all three APIs are added, or each API solution is individually added to its own blend. The tannate salts are mixed with a diluent, flow agents and lubricants. The powder mixture subsequently can be filled into size 1 capsules. A typical capsule formulation prepared by well known conventional encapsulation techniques is shown below.

| Ingredient | mg/capsule |
|---|---|
| carbetapentane tannate* | 60.000 |
| chlorpheniramine tannate** | 4.000 |
| diphenhydramine tannate*** | 25.000 |
| magnesium aluminum silicate, NF | 30.000 |
| Avicel PH 102 | 506.500 |
| Di-Pac (compressible sugar) | 70.000 |
| talc | 2.250 |
| magnesium stearate | 2.250 |

*equivalent to 40 mg carbetapentane citrate
**equivalent to 2.5 mg chlorpheniramine maleate
***equivalent to 12.5 mg diphenhydramine HCl
The ratio of carbetapentane to tannic acid in the tannate salt is 1:1.4, chlorpheniramine to tannic acid is 1:1.3 and diphenhydramine to tannic acid is 1:1, by weight.

The present invention provides a composition comprising diphenhydramine tannate at a consistent purity for the treatment of the symptoms of sneezing, itchy, watery eyes, itchy nose or throat and runny nose due to hay fever (allergic rhinitis) or other respiratory allergies which is superior to compositions containing antihistamine compounds by themselves or in combination.

The compositions of the present invention may contain diphenhdyramine tannate at a consistent purity in the substantial absence of other active ingredients such as other tannate salts. Such compositions are particularly effective for treating symptoms commonly associated with respiratory allergies while avoiding adverse side effects including but not limited to gastrointestinal upsets. Such compositions are particularly useful in treating children as they avoid exposure of the patient to other drugs that are unnecessary to provide effective treatment.

For other applications, the compositions of the present invention may include diphenhydramine tannate at a consistent purity in combination with therapeutic agents from pharmacological classes such as antihistamines, anticholinergics, sympathomimetics, decongestants, cough suppressants, antitussives and expectorants for the treatment of allergic and upper respiratory disorders and symptoms.

Examples of antihistamines that could be used in the combinations include but are not limited to carbinoxamine, chlorpheniramine, pyrilamine, pheniramine, phenindamine, bromodiphenhydramine, triplennamine, cimetidine, ranitidine, and famotidine.

Examples of anticholinergics that could be used in the combinations include but are not limited to methscopolamine, neostigmine and physostigmine.

Examples of antitussives, cough suppressants and expectorants that could be used in the combinations include but are not limited to carbetapentane, dextropmethorphan and guaifenesin.

Examples of decongestants that could be used in the combinations include but are not limited to phenylephrine, pseudoephedrine, ephedrine, cyproheptadine, phenyltoloxamine and clemastine.

Examples of syrnpathomimetics that could be used in the combinations include but are not limited to phenylethylamine, phenylephrine, methoxyphenamine and methoxamine.

Tannic acid may also be used for pH adjustment. Monobasic sodium phosphate, USP, and Dibasic sodium phosphate, USP, Anhydrous may also be included in the formula for pH adjustment.

For the purpose of this disclosure, a warm-blooded animal is a member of the animal kingdom possessed of a homeostatic mechanism and includes mammals and birds.

The dosage administered will be dependent on the age, health and weight of the recipient, kinds of concurrent treatment, if any, frequency of treatment and effect desired. Typically, from about 25 to about 50 mg of the diphenhydramine are administered to adults and children over twelve years of age every four to six hours up to a maximum of about 300 mg in any twenty-four hour period. From about 12.5 to about 25 mg of the diphenhydramine are administered to children from about six to about twelve years of age every four to six hours up to a maximum of about 150 mg in any twenty-four hour period.

In summary, numerous benefits result from the compositions of the present invention. As produced, those compositions are essentially free of contaminates including organic solvents and heat decomposition products including but not limited to benzhydrol, benzophenone, diphenylchloromethane, dimethylaminoethanol, diphenylmethane and diphenyl alkyl ether. The compositions are also characterized by a relatively consistent stoichiometry and potency of active ingredient: that is, the diphenhydramine tannate is provided at a consistent purity. Accordingly, they allow for more precise dosing, a particularly important benefit when used in treating young children.

It should be understood that the above examples are illustrative of the best mode only of the invention herein disclosed. Given the present disclosure, it is anticipated that numerous variations will occur to those skilled in the art. A latitude of modification, substitution and change is intended and in some instances, some features of the invention will be employed without a corresponding use of other features. Accordingly, it is intended that the spirit and scope of the invention disclosed herein should be limited only by the following claims.

1. A therapeutic composition for symptomatic treatment of respiratory allergies in a warm-blooded animal, said composition comprising a pharmaceutically effective amount of diphenhydramine tannate at a consistent purity in substantial absence of an organic solvent and in solid dosage form.

2. The composition of claim 1, wherein said organic solvent is an alcohol.

3. The composition of claim 1, wherein said organic solvent is a mineral oil.

4. The composition of claim 1, wherein said organic solvent is isopropyl alcohol.

5. The composition of claim 1, wherein said organic solvent is glycerin.

6. The composition of claim 1, wherein said organic solvent is propylene glycol.

7. The composition of claim 1, wherein said organic solvent is ethanol.

8. The composition of claim 1, further including at least one additional active ingredient selected from a group consisting of antihistamines, sympathomimetics, decongestants, cough suppressants, antitussives and expectorants.

9. A therapeutic composition for symptomatic treatment of respiratory allergies in a warm-blooded animal, said composition comprising a pharmaceutically effective amount of diphenhydramine tannate at a consistent purity in substantial absence of decomposition products of diphenhydramine produced at temperatures above about 50 degrees C. and in solid dosage form.

10. The composition of claim 9 wherein said decomposition product is benzhydrol.

11. The composition of claim 9 wherein said decomposition product is benzophenone.

12. The composition of claim 9 wherein said decomposition product is diphenylchloromethane.

13. The composition of claim 9 wherein said decomposition product is dimethylaminoethanol.

14. The composition of claim 9 wherein said decomposition product is diphenylmethane.

15. The composition of claim 9 wherein said decomposition product is diphenyl alkyl ether.

16. The composition of claim 9, further including at least one additional active ingredient selected from a group consisting of antihistamines, sympathomimetics, decongestants, cough suppressants, antitussives and expectorants.

17. A therapeutic composition for symptomatic treatment of respiratory allergies in a warm-blooded animal, comprising a pharmaceutically effective amount of diphenhydramine tannate at a consistent purity prepared by:
(a) dissolving the salt or free base of the diphenhydramine in a pharmaceutically acceptable liquid to form a solution at a maximum temperature and pH value, that does not cause decomposition of the active pharmaceutical ingredient;
(b) separately mixing an anti-clumping agent with tannic acid to generate a blend;
(c) combining the solution from step (a), with the blend of step (b) to form a tannate salt of diphenhydramine;
(d) combining the tannate salt of the diphenhydramine of step (c) with a pharmaceutically acceptable excipient to form a granulate; and
(e) processing the granulate into a tablet, capsule or other solid dosage form.

18. The composition of claim 17, further including at least one additional active ingredient selected from a group consisting of antihistamines, sympathomimetics, decongestants, cough suppressants, antitussives and expectorants.

19. A method for symptomatically treating respiratory allergies in a warm-blooded animal, comprising administering to said warm-blooded animal a pharmaceutically effective amount of diphenhydramine tannate at a consistent purity in substantial absence of an organic solvent and in solid dosage form.

20. A method for symptomatically treating respiratory allergies in a warm-blooded animal, comprising administering to said warm-blooded animal a pharmaceutically effective amount of diphenhydramine tannate at a consistent purity in substantial absence of decomposition products of diphenhydramine produced at temperatures above about 50 degrees C. and in solid dosage form.

21. A method for symptomatically treating respiratory allergies in a warm-blooded animal, comprising administering to said warm-blooded animal a pharmaceutically effective amount of diphenhydramine tannate at a consistent purity prepared by:
(a) dissolving the salt or free base of the diphenhydramine in a pharmaceutically acceptable liquid to form a solution at a maximum temperature and pH value, that does not cause decomposition of the active pharmaceutical ingredient;
(b) separately mixing an anti-clumping agent with tannic acid to generate a blend;
(c) combining the solution from step (a), with the blend of step (b) to form a tannate salt of diphenhydramine;
(d) combining the tannate salt of the diphenhydramine of step (c) with a pharmaceutically acceptable excipient to form a granulate; and
(e) processing the granulate into a tablet, capsule or other solid dosage form.

22. The composition of claim 1 in substantial absence of any other active ingredient.

23. The composition of claim 1 in substantial absence of any other tannate salt.

24. The composition of claim 9 in substantial absence of any other active ingredient.

25. The composition of claim 9 in substantial absence of any other tannate salt.

26. The composition of claim 17 in substantial absence of any other active ingredient.

27. The composition of claim 17 in substantial absence of any other tannate salt.
